(19) United States
(12) Patent Application Publication
(10) Pub. No.: US 2024/0324931 A1
(43) Pub. Date: Oct. 3, 2024

(54) ELECTROCARDIOGRAPHIC IMAGING SYSTEM AND METHOD

(71) Applicant: UCL Business Ltd, London (GB)

(72) Inventors: Gabriella Captur, London (GB); George Joy, London (GB); Matthew Webber, London (GB); Pier Lambiase, London (GB); Michele Orini, London (GB)

(73) Assignee: UCL Business Ltd, London (GB)

(21) Appl. No.: 18/194,235

(22) Filed: Mar. 31, 2023

(51) Int. Cl. A61B 5/282; A61B 5/00; A61B 5/274

(52) U.S. Cl. CPC A61B 5/282; A61B 5/274; A61B 5/6805; A61B 5/6831; A61B 2562/046

(57) ABSTRACT

Disclosed is an electrocardiographic imaging (ECGi) system including an electrode vest (100) that comprises a flexible base layer (110) configured to cover at least a patient's chest area; and a plurality of dry contact electrodes (120) attached to the flexible base layer (110) and distributed over at least a chest area of the flexible base layer (110) to acquire electrophysiological signals from the patient's skin surface. Each electrode (120) comprises a sensing portion having an exposed sensing surface on an interior skin facing side of the electrode to contact the user's skin surface when the vest is worn, and an exposed connector portion on an exterior side of the electrode for removably connecting with an electrical lead of an ECGi measurement apparatus.

### TECHNICAL FIELD

This invention relates generally to electrocardiographic imaging, particular to electrode vests for implementing the same in combination with cardiovascular magnetic resonance (CMR) imaging.

### BACKGROUND

Arrhythmias are abnormal electrical heart rhythms that occur in patients with cardiovascular disease (CVD). Arrhythmias can be life-threatening, thus clinicians may recommend that patients receive an implantable cardiac defibrillator (ICD) which will deliver a life-saving shock if an arrhythmia occurs. Though potentially life-saving, the side-effects of ICDs can have a profound negative impact on patient quality of life. For example, the ICD can sometimes transmit unnecessary shocks or get infected or displaced, and anxiety and depression are commonly reported. Due to these complications, clinicians must carefully select which patients should receive an ICD, using information on the electrical activity and structure of the patient's heart as guidance.

In clinical practice, information on the electrical activity of the heart is typically collected using a standard 12-lead electrocardiogram (ECG), however this can miss subtle abnormalities and lacks the spatial resolution to locate the origin of an arrhythmia.

Electrocardiographic imaging (ECGi) is an emerging technology that maps the electrical activity of the heart using 256 electrodes across the chest area to measure the body surface potentials, providing more diagnostic data than standard 12-lead ECGs. ECGi combines this high-density electrophysiological ECG data, with imaging-derived heart-torso geometries (structural data), to produce electrical maps of the heart's surface. These electrical maps can help clinicians determine which structural changes are causative of arrhythmias, and thus determine the risk of a patient experiencing further arrythmias and guide treatment.

Information on the structure of the heart for merging with ECGi can be collected using one of two techniques: computerized tomography (CT) or cardiovascular magnetic resonance (CMR) imaging. CMR is superior to CT, as it is radiation free and able to provide information on abnormal tissue characteristics and inform in a pixelwise manner about regions of myocardial inflammation, fibrosis, fatty infiltration, protein deposition, or impaired perfusion, all of which is not possible by CT. Combining ECGi with CMR can therefore help to better understand the arrhythmogenic potential of changes seen on CMR, and therefore further improve the clinician's ability to predict how “at risk” a patient is from life-threatening arrhythmias. However, there is currently no re-usable and cost-effective technology available for combining ECGi with CMR, which has prevented this approach from being developed further.

State of the art ECGi systems include the Medtonic “CardioInsight” system, and the BioSemi ECGi system. The Medtronic system uses a single-use (disposable) wearable vest comprising embedded “dry” contact silver/silver chloride electrodes and metal leads to capture electrical signals from the body surface, and combines these signals with CT scan data to produce and display simultaneous, bi-atrial and biventricular, 3-D cardiac maps. The patient wears the vest during and after the CT scan, which occurs immediately prior to ECG data collection so that the ECG data correlates with the CT scan data. The vest cannot be washed due to the embedded metal components and so is disposed of after use due to potential transmission of infection. It is also not CMR compatible because it is composed of materials that heat up in the strong magnetic fields of a CMR scanner, potentially causing skin burns. The BioSemi ECGi system combines ECG data with CMR. It uses a multitude of adhesive “wet” electrode-containing strips, that are stuck onto the patient's chest. The strips contain carbon electrodes and an integrated pre-amp at the end of the strip. Once attached, each of the strips are then filled with a conductive gel using a syringe to interface with the skin. After ECG data collection, the electrode strips are removed from the patient, cleaned of the gel and replaced by cod liver oil markers or other MR-lucent fiducial markers for visualisation during CMR. These markers indicate the electrodes prior position for aligning the electrophysiological ECG data to the structural data obtained during CMR. However, although the BioSemi electrode strips are reusable after cleaning, they are costly to manufacture, they are time consuming to prepare, attach, remove and clean, the use of gel increases the risk of short circuiting arising from a high concentration of electrodes, and their adhesive-backed nature and necessary removal prior to CMR imaging can cause discomfort to the patient.

There is therefore a need for re-usable, simple and cost-effective CMR compatible technology for combining ECGi with CMR. Aspects and embodiments of the present invention have been devised with the foregoing in mind.

### SUMMARY OF THE INVENTION

According to an aspect of the present invention, there is provided an electrocardiographic imaging (ECGi) electrode vest. The vest is configured to be used in conjunction with cardiovascular magnetic resonance (CMR) imaging, but may be used with other imaging modalities, such as computerized tomography (CT). The vest comprises a flexible base layer configured to cover at least a patient's chest area; and a plurality of dry contact electrodes attached to the flexible base layer and distributed over at least a chest area of the flexible base layer to acquire electrophysiological signals from the patient's skin surface. Each electrode comprises a sensing portion having an exposed sensing surface on an inner skin facing side of the electrode to contact the user's skin surface when the vest is worn, and an exposed connector portion on an opposite/exterior side of the electrode for removably connecting with an electrical lead of an ECGi measurement apparatus. The vest is preferably washable, re-useable and CMR compatible.

The exposed connector portions are accessible from the exterior side of the vest (when worn), such that each electrode can be individually connected to an external electrical lead for transmitting the signal to the ECGi measurement apparatus. This means the vest does not include any integrated or embedded metal electrical leads or traces connected to the electrodes for conducting the electrophysiological signals to another location, e.g. to one or more connectors, which in turn makes the vest entirely washable and thereby re-useable. That is, the electrical leads can be disconnected after use for washing. It also means that the vest is compatible with cardiovascular magnetic resonance (CMR) imaging, because the electrical leads can be disconnected from each electrode prior to performing CMR imaging.

The electrodes are formed of or comprise one or more conductive, but washable materials. For example, the conductive material is a solid material (as opposed to a gel or the like). In embodiments, at least part of the electrodes are formed of or comprise a conductive polymer material. Alternatively or additionally, at least part of the electrodes may be formed of or comprise a metallic material. The electrodes may comprise a combination of metal and conductive polymer portions/parts. At least part of the electrodes may be or comprise a solid body. Alternatively or additionally, at least part of the electrodes may be or comprise a conductive fabric.

Preferably, the connector portion comprises a protrusion for removably connecting with an electrical lead of an ECGi measurement apparatus. The protrusion forms a male connector element for releasably engaging a female connector element of the electrical lead. The protrusion may include one or more ridges or depression on, in or around a transverse wall of the protrusion for facilitating interlocking mechanical engagement with an electrical lead.

Preferably, the base layer comprises a plurality of openings co-located with the electrodes. Each electrode extends through a respective one of the plurality of openings and comprises first and second flanges that extend over opposite sides of the base layer to grip or hold the base layer therebetween.

Preferably, each electrode comprises an electrode body including first part and a second part. The electrode body may be formed of or comprise a conductive polymer material or a metal. The first part includes the sensing portion and the first flange, and the second part includes the connector portion and the second flange. The first and second parts are connectable to each other through a respective opening in the base layer to attach the electrode body to the base layer. Optionally or preferably, the first and second parts are configured to connect in a snap-fit manner so as to maintain their attachment. In this case, the first and second parts comprise integral interlocking attachment features, such as depressions and ridges etc.

Optionally, each electrode can further comprise a third electrode part disposed on or embedded in a skin facing side of the base layer adjacent the respective opening. The third part has an exposed sensing surface to contact the user's skin surface when the vest is worn, and is in electrical contact with the electrode body when the first and second parts are connected through the respective opening. The third electrode part thereby serves to extend the sensing surface area of the electrode. The third electrode part may be formed of or comprise a conductive polymer material or a metal. The third electrode part may comprise a conductive layer or conductive fabric.

Preferably, the electrodes are uniformly distributed in an array over at least the chest area of the base layer.

Optionally, the vest further comprises a plurality of fiducial markers that are substantially co-located with the plurality of electrodes. The fiducial markers are magneto-resonance (MR) lucent and visible in a CMR image, allowing structural data obtained from CMR imaging to be aligned with the ECG data obtained from the electrode vest. The fiducial markers may include a plastic capsule or packet filled with an MR-lucent material (e.g. a fluid). The fiducial markers are preferably designed and configured for optimum patient comfort. The fiducial markers may have a size or width of 2 cm or less, preferably, 1 cm or less.

The flexible base layer may further be configured to cover the user's back area. In this case, the plurality of electrodes may comprise a first group of electrodes attached to and distributed across the chest area of the flexible base layer; and a second group of electrodes attached to and distributed across a back area of the flexible base layer.

Alternatively, the flexible base layer may be a front base layer, and the vest further comprises a flexible rear base layer to cover the user's back area. In this case, the plurality of electrodes may comprise a first group of electrodes attached to and distributed across the chest area of the front base layer; and a second group of electrodes attached to and distributed across a back area of the rear base layer.

The electrode vest may comprise 128 or 256 electrodes. Where the vest covers the back area or comprises a rear base layer, the first and second groups of electrodes may each include 128 electrodes.

The flexible base layer may be formed of or comprises one of a: woven synthetic fabric, non-woven synthetic fabric, polyester-elastane material.

According to another aspect of the invention, there is provided an electrocardiographic imaging (ECGi) kit. The kit comprises the ECGi electrode vest with any combination of the features described above; and an inflatable gilet or vest configured to be worn over the electrode vest during electrophysiological signal acquisition to improve the skin-electrode contact. The inflatable gilet is configured to cover at least the chest area of the electrode vest, and, when worn over the electrode vest and inflated, to exert a compressive force on the electrode vest to bias the electrodes towards a patient's skin surface.

The kit may further include a fiducial marker vest. The fiducial marker vest comprises a flexible base layer configured to cover at least a patient's chest area, and a plurality of fiducial markers distributed over a chest area of the base layer in the same locations as the electrodes of the electrode vest. The fiducial marker vest is CMR compatible and configured for wearing during CMR imaging in the place of the electrode vest. The fiducial markers are (MR) lucent and visible in a CMR image, allowing structural data obtained from CMR imaging to be aligned with the ECG data obtained from the electrode vest. The fiducial markers may include a plastic capsule or packet filled with an MR-lucent material (e.g. a fluid). The fiducial markers are preferably designed and configured for optimum patient comfort. The fiducial markers may have a size or width of 1 cm or less. In this sense, the CMR compatible marker vest does not include any metal components. Further, the fiducial marker vest is washable and thereby re-useable. Preferably, the size and shape of the base layer of the marker vest is identical to the size and shape of the electrode vest. Preferably, the base layers of the electrode vest and the marker vest are comprised of the same material(s).

The kit may further comprise a plurality of electrical leads for connecting to the electrodes of the electrode vest. Each electrical lead comprises an electrode connector head at one end of thereof that is configured to removably connect with a respective connector portion of a respective electrode of the electrode vest for transmitting electrophysiological signals to an ECGi measurement apparatus. Each lead may include a connector at the other end for connecting to the ECGi measurement apparatus.

According to yet another aspect of the invention, there is provided an electrocardiographic imaging (ECGi) system. The ECGi system comprises the ECGi electrode vest with any combination of the features described above, and an ECGi measurement apparatus for measuring the ECG signals. The measurement apparatus may comprise a data acquisition system with a plurality of measurement channels, and a plurality of electrical leads. Each electrical lead may comprise an electrode connector head at one end thereof, the electrode connector head configured to removably connect to a respective connector portion of a respective electrode of the electrode vest for transmitting electrophysiological signals to the respective measurement channels of the data acquisition system.

Preferably, each connector portion of each electrode comprises a protrusion, and each electrode connector head comprises a mechanical clip to releasably engage the protrusion, and/or a female connector configured to receive and frictionally and/or mechanically engage the protrusion. Preferably, the electrode connector head and the protrusion are configured to releasably connect in a snap-fit manner so as to maintain their attachment. In this case, the electrode connector head and the protrusion may comprise integral interlocking attachment features, such as depressions and ridges etc. Optionally, each electrode connector head comprises a signal pre-amplifier.

The system may further comprise an inflatable gilet configured to be worn over the electrode vest during electrophysiological signal acquisition to improve the skin-electrode contact.

The system may comprise any combination of features of the ECGi kit described above.

According to yet another aspect of the invention, there is provided a method of electrocardiographic imaging (ECGi). The method comprises acquiring electrophysiological signals from a plurality of locations on the chest skin surface of a patient using the electrode vest that includes any combination of the features described above. This step may obtain ECG data. The method further comprises acquiring structural information or data of the patient's heart by performing cardiovascular magnetic resonance (CMR) imaging on the patient. The method may further comprise generating one or more maps of electrical activity of the patient's heart based on the electrophysiological signals and the structural information. The one or more maps may include a bi-atrial map, a biventricular map, and/or a three-dimensional cardiac map.

Preferably, the step of acquiring electrophysiological signals comprises fitting the electrode vest to the patient; fitting an inflatable gilet over the electrode vest; and inflating the inflatable gilet to bias the electrodes of the electrode vest towards the patient's skin surface.

The step of acquiring electrophysiological signals may further comprise attaching a plurality of electrical leads to the electrodes.

Optionally, where the electrode vest comprises a plurality of fiducial markers substantially co-located with the plurality of electrodes, the step of acquiring structural information may comprise: removing the electrical leads from the electrode vest prior to performing CMR imaging; and performing CMR imaging on the patient while the patient wears the electrode vest, wherein the fiducial markers are captured by the CMR imaging. The step of generating the one or more maps of electrical activity of the patient's heart based on the electrophysiological signals and the structural information may then comprise aligning the electrophysiological signals (or ECG data) obtained from the electrodes of the electrode vest to the structural information using the captured fiducial markers in the CMR imaging.

Optionally, the step of acquiring structural information comprises: removing the electrode vest and fitting a fiducial marker vest to the patient, the fiducial marker vest comprising a flexible base layer configured to cover at least the patient's chest area, and a plurality of fiducial markers distributed over the chest area of the base layer in the same locations as the electrodes of the electrode vest; and performing CMR imaging on the patient, wherein the fiducial markers are captured by the CMR imaging. The fiducial marker vest is preferably washable and re-usable. The step of generating the one or more maps of electrical activity of the patient's heart based on the electrophysiological signals and the structural information may then comprise: aligning the electrophysiological signals obtained from the electrodes of the electrode vest to the structural information using the captured fiducial markers in the CMR imaging.

A vest is generally defined herein as a garment configured to be worn over the torso that covers at least the chest area, and preferably the back area of a patient. For example, a vest may include front and rear panels/base layers that are connected/connectable by shoulder and side straps.

Features which are described in the context of separate aspects and embodiments of the invention may be used together and/or be interchangeable. Similarly, where features are, for brevity, described in the context of a single embodiment, these may also be provided separately or in any suitable sub-combination. Features described in connection with the ECGi electrode vest, kit or system may have corresponding features definable with respect to the method, and vice versa, and these embodiments are specifically envisaged.

### BRIEF DESCRIPTION OF THE DRAWINGS

In order that the invention can be more fully understood, embodiments will now be discussed in the following detailed description by way of example only with reference to the accompanying drawings, in which:

FIG. 1 shows a schematic block diagram of an electrocardiographic imaging (ECGi) system according to an embodiment of the present disclosure;

FIG. 2 shows a schematic diagram of an ECGi electrode vest of the ECGi system according to an embodiment of the present disclosure;

FIG. 3 shows a schematic sectional view of the ECGi electrode vest in FIG. 1 taken along line 3-3;

FIG. 4 shows a schematic sectional view of an electrode of the ECGi electrode vest according to an embodiment of the present disclosure;

FIGS. 5A-5D show schematic sectional views of an electrode of the ECGi electrode vest comprising first and second parts according to different embodiments of the present disclosure;

FIGS. 6A and 6B shows schematic sectional views of an electrode of the ECGi electrode vest comprising first, second and third electrode parts according further embodiments of the present disclosure;

FIG. 7 shows a schematic block diagram of an electrode connector head of an electrical lead for connecting to an electrode of the ECGi electrode vest;

FIG. 8A shows a schematic diagram of an example electrode connector head of an electrical lead for connecting to an electrode of the ECGi electrode vest via frictional engagement;

FIG. 8B shows a schematic diagram of an example electrode connector head of an electrical lead for connecting to an electrode of the ECGi electrode vest via mechanical engagement;

FIGS. 9A and 9B show images of an example ECGi electrode vest fitted to a patient;

FIG. 10 shows a schematic diagram of an inflatable gilet for wearing over the electrode vest;

FIG. 11 shows a schematic sectional view of the inflatable gilet in FIG. 10 taken along line 11-11;

FIG. 12 shows an image of the ECGi electrode vest and inflatable gilet of FIG. 11 fitted to a patient;

FIG. 13 shows a schematic diagram of an ECGi electrode vest of the ECGi system including fiducial markers according to an embodiment of the present disclosure;

FIG. 14A shows a schematic diagram of a fiducial marker vest for wearing in a CMR scanner according to an embodiment of the present disclosure;

FIG. 14B shows a schematic sectional view of the fiducial marker vest in FIG. 13A taken along line 14B-14B; and

FIG. 15 shows a ECGi method according to an embodiment of the present disclosure.

It should be noted that the figures are diagrammatic and may not be drawn to scale. Relative dimensions and proportions of parts of these figures may have been shown exaggerated or reduced in size, for the sake of clarity and convenience in the drawings. The same reference signs are generally used to refer to corresponding or similar features in modified and/or different embodiments.

### DETAILED DESCRIPTION

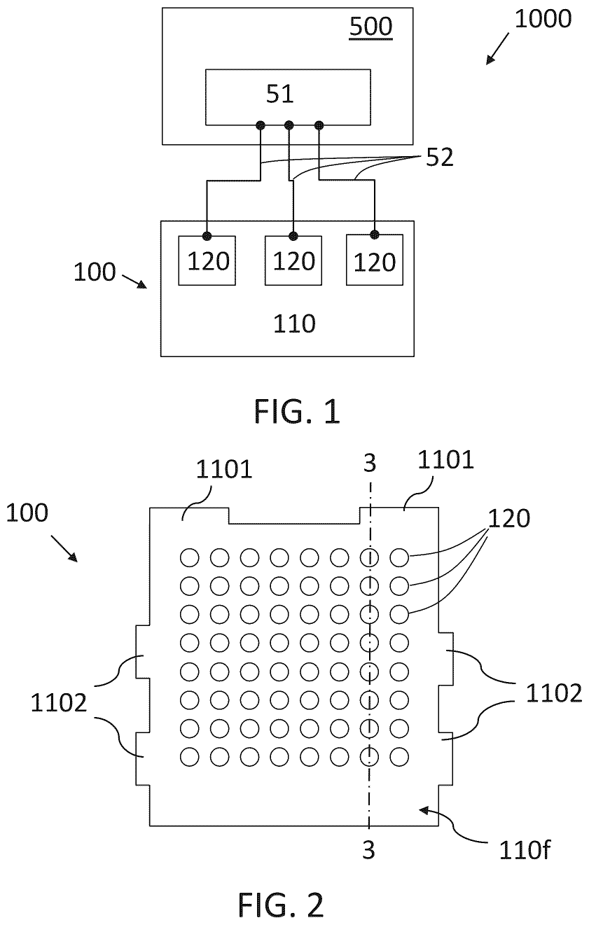

FIG. 1 is a schematic block diagram of an electrocardiographic imaging (ECGi) system 1000, according to an embodiment of the present disclosure. The ECGi system 1000 comprises a wearable electrode vest 100 including a plurality of “dry” contact electrodes 120 for acquiring electrophysiological (ECG) signals from a patient's skin surface, and a ECGi measurement apparatus 500 to measure the ECG signals from the electrodes 120. The vest 100 comprises a flexible base layer 110 configured to cover at least a patient's chest area. The plurality of electrodes 120 are attached to the flexible base layer 110 and distributed over at least a chest area of the flexible base layer 110. Although only three electrodes 120 are shown for clarity, it will be appreciated that in practice the vest 100 will include a larger number of electrodes 120. For example, in various embodiments the electrode vest 100 comprises 256 electrodes 120 (see below). The measurement apparatus 500 comprises a data acquisition system 51 with a plurality of measurement channels, one for each electrode 120, and a signal amplifier. In one implementation, the data acquisition system 51 may further include one or more processing devices and a display, the one or more processing devices configured to execute a ECG signal visualisation and recording program. Each electrode 120 is individually removably connectable to a measurement channel of the data acquisition system 510 by a respective electrical lead 52. The data acquisition system 51 is configured to measure the electrical voltage or potential at each electrode simultaneously with low noise and high resolution and a sampling rate suitable for bio-signals. In one example, the ECG signals are measured at rest or during exercise at a sampling rate of 2400 Hz and with sensitivity range from −10 mV to +10 mV. The measurement apparatus 500 may also receive a reference signal from a ground electrode that is separately attached to the patient's chest, e.g. via a disposable stick-on electrode.

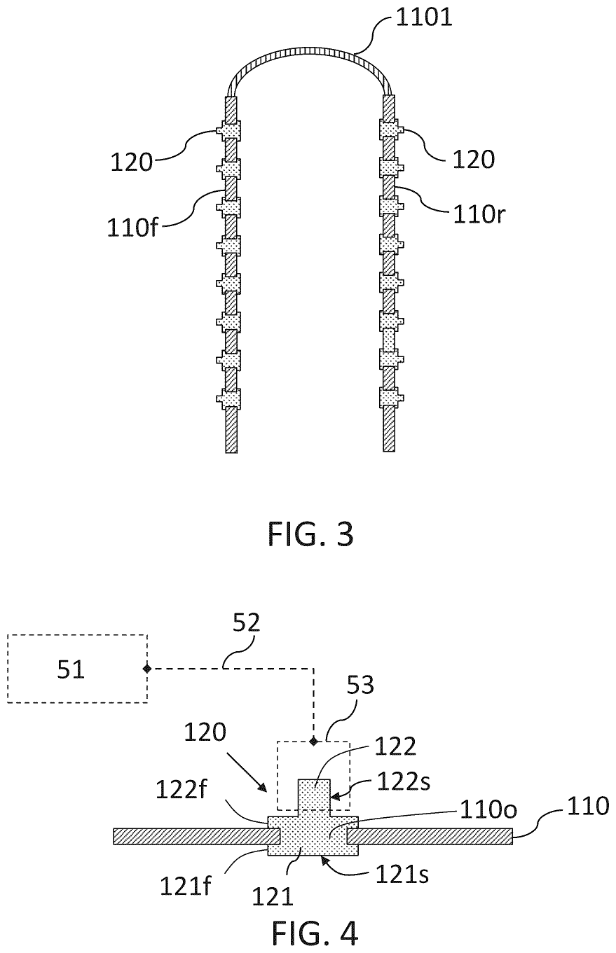

FIG. 2 shows a schematic front view of an exemplary electrode vest 100, and FIG. 3 shows a schematic sectional view of the electrode vest 100 taken trough line 3-3. The electrode vest 100 is a garment dimensioned and configured to be worn or applied over a torso of a patient (a human patient) that covers at least part of the front and back of the patient's torso. Different configurations can be utilised depending on the patient (e.g. the human or other animal). The electrode vest 100 can be made in a variety of sizes to accommodate a range of patient's sizes and body types. However, a key feature of the electrode vest 100 of the invention is the electrode structure and materials used which makes it washable and re-usable, as well as compatible with cardiovascular magnetic resonance (CMR) imaging (as will become apparent in the following description).

The electrode vest 110 comprises flexible front and rear base layers 110f, 110r that are connected via adjustable shoulder straps 1101 and adjustable side straps 1102, as shown. The front base layer 110f covers the patient's chest area, and the rear base layer 110r covers the back area and are secured onto the patient using the adjustable straps 1101, 1102. The base layers 110f, 110r provide an electrode-carrying substrate layer. A first group of the plurality of electrodes 120 are attached to, and distributed over, the chest area of the front base layer 110f, and a second group of the plurality of electrodes 120 are attached to, and distributed over, the back area of the rear base layer 110r. The electrodes 120 are preferably uniformly distributed in an array across the chest and back areas, as shown. Although shown as a square array, it will be appreciated that the electrodes 120 can be distributed in various other array geometries, e.g. a triangular array, hexagonal array, or interleaved array (not shown). Further, the vest 100 may instead comprise a plurality of electrodes array sections, or sub-groups of electrodes 120, dimensioned and configured for positioning over different portions of the patient's torso.

The front and rear base layers 110f, 110r are preferably formed of a single panel of flexible and washable material, such as a natural or synthetic fabric or a plastic/polymer, to which the adjustable staps 1101, 1102 and electrodes 120 are attached. However, it will be appreciated that the front and rear base layers 110f, 110r can instead be formed of multiple panels of material that are connected or stitched together. Suitable materials for the base layers 110f, 110r include but are not limited to: a cotton-based material, a linen-based material, a polyamide-based material, a nylon-based material, a polyester-based material, a cotton-polyester blend, a polyester-elastane material, a spandex-based material, and a polypropylene-based material.

The straps 1101, 1102 preferably comprise a releasable and adjustable fastener such as hook and loop fastener to enable easy fitting and removal of the vest 100, e.g. without removal of the electrical leads 52 if necessary (discussed in more detail below).

The electrodes 120 are formed or comprise one or more conductive materials. Preferably, the electrode materials are washable and non-toxic (to avoid skin irritation). In some embodiments, at least part of the electrodes 120 are formed of or comprise a mouldable or thermo-formable conductive polymer material. The conductive polymer material can be substantially rigid, soft, deformable and/or flexible depending on its composition. In embodiments, the conductive polymer material comprises a polymer base material mixed with or containing a proportion of conductive filler material such as conductive powder, particles, fibers chemical formulation or other materials as is known the art. The conductive filler material is preferably a carbon-based material, such as graphite, pseudo graphite, graphene, graphene-oxide, carbon black, carbon fiber, carbon nanotubes or carbonized fabric, but may instead be a metal-based material such as copper, copper nanoparticles, silver, aluminum, titanium oxide, etc. Suitable polymer base materials include but are not limited to: silicone rubber; thermoplastic polyurethane, thermoplastic polyethylene, thermoplastic polypropylene, thermoplastic elastomer, acrylonitrile butadiene styrene, poly(3,4-ethylenedioxythiophene), and poly(styrenesulfonate). The conductive polymer material can have a range of hardness properties (e.g. depending on the base material) with a controllable conductivity (e.g. depending on the conductive material content or graphite ratio).

In other embodiments, at least part of the electrodes 120 are formed of or comprise a metallic material, such as silver or silver chloride. The electrodes 120 may comprise a conductive thread (e.g. metal or silver plated polyamide/elastomer yarn) or a chemical formulation that is printed onto the base layer 110f, 110r or applied to the base layer 110f, 110r, e.g. by drip-coating.

FIG. 4 shows a schematic sectional view of an electrode 120. Each electrode 120 operates as a sensor for detecting electrical activity on the body surface (body surface potentials) and providing a corresponding electrical (ECG) signal. Although depicted as generally circular in FIG. 4, other shapes can be utilized in other examples (e.g. square, rectangular, oval, polygon or the like). The electrodes 120 are “dry” contact electrodes, which are configured to operate without the use of a conductive gel interface or adhesive. That is, by direct contact between the electrode 120 and the skin surface (in contrast to “wet” electrodes which require a conductive gel interface between the skin surface and electrode, as is known the art). The electrode 120 comprises a sensing portion 121 and a connector portion 122. The sensing portion 121 includes an exposed sensing surface 121s on an inner skin-facing side of the electrode 120 to contact the patient's skin surface when the vest 100 is worn and detect the electrical activity. The connector portion 122 includes a projection with exposed connector surface 122s on an opposite (outer/exterior) side of the electrode 120 for providing the electrical ECG signal. The exposed connector surface 122s of the connector portion 120 is accessible from the exterior of the vest 100 for removably connecting with an electrical lead 52 of an ECGi measurement apparatus 500 to transmit the electrical signal from the electrode 120 to the ECGi measurement apparatus 500.

As can be seen, the vest 100 does not include any integrated or embedded electrical traces, leads or wires for transmitting the signal from the electrode to another location, such as a separate connector. Only external electrical leads 52 are required to make the connection to the measurement apparatus 500. Each electrical lead 52 comprises an electrode connector head 53 at one end of thereof configured to removably connect with a respective connector portion 122 and transmit the ECG signals to the data acquisition system 51, as indicated by the dashed lines in FIG. 4. In one example implementation, the connector portion 122 and the electrode connector head 53 are configured with interlocking features to provide a snap-fit connection (see below).

With reference again to FIG. 4, the front and rear base layers 110f, 110r each comprise a plurality of openings 110o through which the electrodes 120 extend. The exposed connector portion 120 is located on and accessible from the exterior side of the vest 100 for connecting the electrical leads 52, and the sensing portion 121 is located on the interior skin-facing side of the vest 100 for contacting the skin. To attach to the base layer 110f, 110r, each electrode 120 comprises first and second flanges 121f, 122 that extend over opposite sides of the base layer 110 to grip the base layer 110f, 110r therebetween. The first flange 121f extends over the inner skin-facing side of the base layer 110f, 110r and the second flange 122f extends over the outer/exterior side of the base layer 110f, 110r.

In preferred embodiments, the electrode 120 comprises a main electrode body formed of a first part and a second part that are connectable to each other to secure the electrode 120 to the base layer 110f, 110r. The first part includes the sensing portion 121 and the first flange 121f, and the second part 122 includes the connector portion 122 and the second flange 122f. The first and second parts 121, 122 are configured to connect to each other through a respective opening 110o in order to grip the base layer 110f, 110r between the first and second flanges 121f, 122. In one example implementation, the first and second parts take the form of male and female elements that are configured to engage with each other in a snap-fit manner, as is known in the art. The first and second parts 121, 122 are preferably formed of or comprise a thermo-formable conductive polymer as described above, but may instead be formed of a metal material.

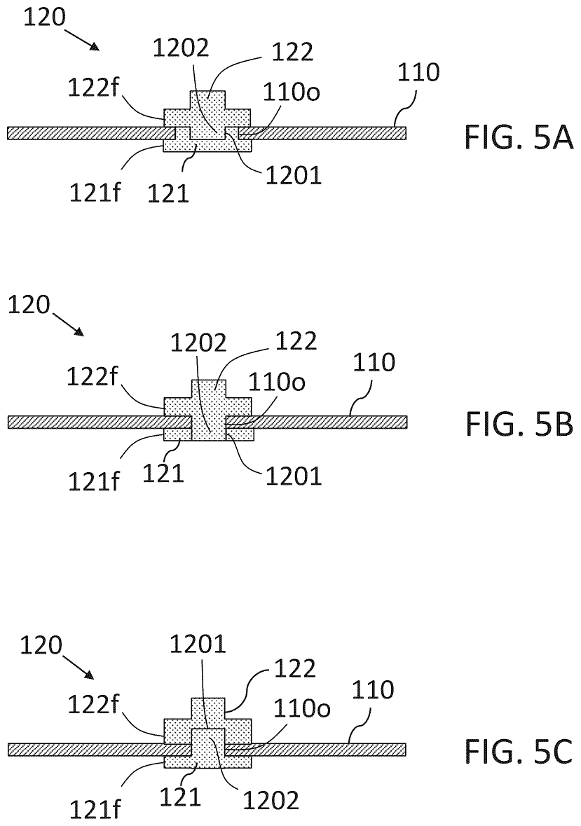

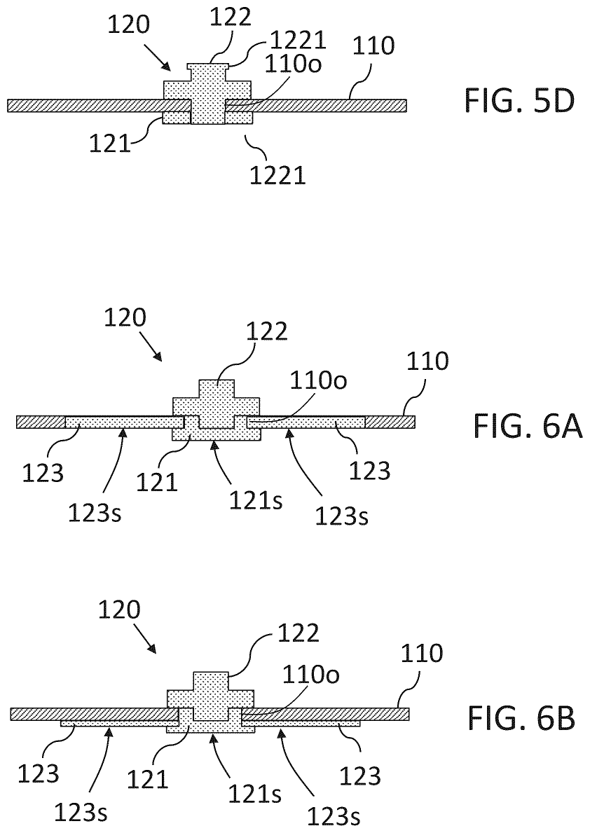

FIG. 5A shows a schematic sectional view of an electrode 120 comprising first and second parts 121, 122 according to an example implementation. In this example, the second part 122 is a male element comprising a projection 1202 extending towards to the first part 121, and the first part 121 is a female element comprising a recess or opening 1201 configured to receive and engage the projection 1202 of the second part 122. The opening 1201 can extend partially through the first part 121 as shown in FIG. 5A, or it can extend fully through the first part 121 as shown in FIG. 5B. In an alternative configuration, the second part 122 can be the female element comprising the recess or opening 1201 and the first part 121 can be the male element comprising the projection 1202, as depicted in FIG. 5C.

When connected, the male and female elements can be secured by frictional engagement alone (i.e. an interference fit) or by mechanical (interlocking) engagement. In a preferred embodiment, the first and second parts 121, 122 are secured, when connected, by mechanical engagement. For example, taking the projection 1202 and opening 1201 in a longitudinal direction, the lateral/transverse walls of the recess 1201 and projection 1202 can comprise interlocking features, such as a ridge/detent and depression/notch, to provide a snap-fit connection and retain the first and second parts 121, 122 in a connected state.

In embodiments, the outwardly extending projection of the connector portion 122 can include a one or more ridges or notches facilitating a mechanical snap fit connection with the electrode connector head 53, as depicted in FIG. 5D.

In addition to the first and second parts 121, 122 of the electrode body described above, in various embodiments the electrodes 120 further comprise a third electrode part or sensor pad 123 disposed on or embedded in a skin facing side of the base layer 110f, 110r adjacent the respective opening 110o, as shown in FIGS. 6A and 6B. The third electrode part 123 includes an exposed sensing surface 123s to contact the user's skin surface when the vest 100 is worn, and is in electrical contact with the electrode body when the first and second parts 121, 122 are connected through the respective opening 110o. In this way, the third electrode part or sensing pad 123 serves to extend the area of the sensing surface of the electrode 120, thereby increasing the signal-to-noise ratio of the measured ECG signal.

In one implementation, the third electrode part or sensing pad 123 comprises a thermo-formable conductive polymer material, similar to the electrode body 121, 122. The conductive polymer material can be printed or coated onto the skin facing side of the base layer 110f, 110r, or embedded into the fabric of the base layer 110f, 110r, e.g. by heat pressing or drip-coating. Alternatively, the third electrode part 123 can comprise a conductive yarn or fiber woven into the base layer 110f, 110r, such as a silver or silver coated yarn.

The non-metallic conductive polymer electrode body and fabric base layers 110f, 110r, combined with the absence of integrated metal electrical leads or wiring for carrying the ECG signals from the electrodes 120 (e.g. to a common connector), makes the electrode vest 100 entirely washable and thereby re-usable. Further, it means the electrode vest 100 is compatible with cardiovascular magnetic resonance (CMR) imaging and can be worn during CMR imaging. For example, once ECG measurements are taken, the electrical leads 52 can be disconnected from the electrodes 120, and the vest 100 worn during CMR imaging, as described in more detail below.

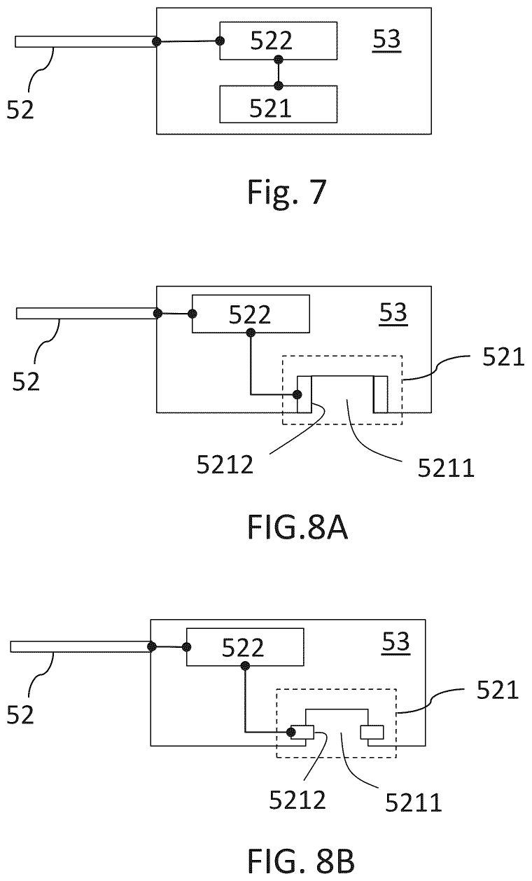

FIG. 7 shows a schematic diagram of an electrode connector head 53 of an electrical lead 52. The electrode connector head 53 comprises a female connector portion 521 configured to releasably engage and connect with the projection of the electrode connector portion 122. The electrode connector head 53 may optionally include an integrated signal pre-amplifier to improve signal-to-noise ratio. With reference to FIG. 8A, in one example implementation, the female connector portion 521 comprises an opening 5211 to receive the projection of the electrode connector portion 122 and electrical contacts 5212 configured to frictionally engage a wall of the projection 122 and establish an electrical connection therebetween. The electrical contacts 5212 may be formed of or comprise a metal or conductive polymer material (such as any of those described above). Alternatively or additionally, the electrical contacts 5212 can be configured to mechanically (interlockingly) engage a wall of the projection 122 to establish an electrical connection therebetween and provide a releasable snap-fit or snap on connection. For example, the electrical contacts 5212 can form or include a ridge or a clip as depicted in FIG. 8B that interlocks with one or more ridges or depressions on the wall of the projection of the connector portion 122. An mechanical or snap-on type connection advantageously may allow the electrode connector head 53 to rotate about the projection of the connector portion 122 whist maintaining a stable electrical connection. This may allow adjustment of the electrical leads 52 once connected.

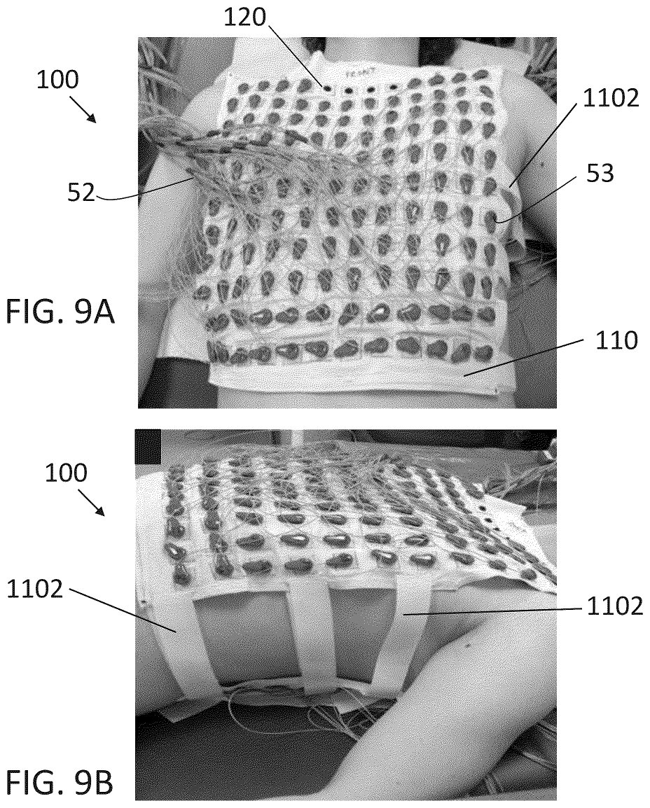

FIGS. 9A and 9B show images of an example electrode vest 100 fitted to a patient, with electrical leads 52 connected to each individual electrode 120.

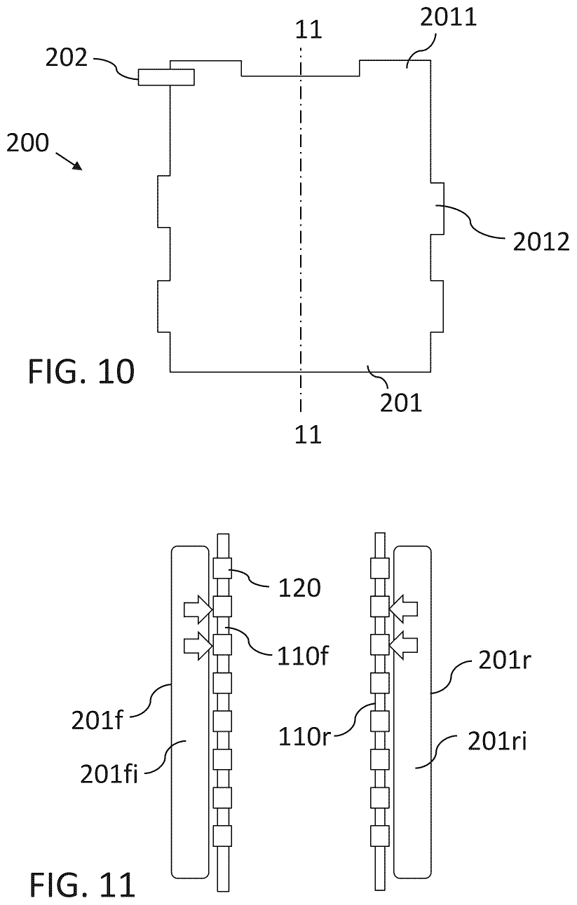

FIG. 10 shows a schematic front view of an inflatable gilet or vest 200 for use with the electrode vest 110. FIG. 11 shows a schematic sectional view of the inflatable gilet 200 taken along line 11-11. The inflatable gilet 200 is configured to be worn over the electrode vest 100 during electrophysiological signal acquisition to improve the skin-electrode contact. Specifically, the inflatable gilet 200 is configured to cover at least the electrodes 120 on the chest and back areas of the electrode vest 100, and, when inflated, to exert a compressive force on the electrodes 120 of the vest 100 to bias the electrodes 120 towards a patient's skin surface, as indicated by the arrows in FIG. 11.

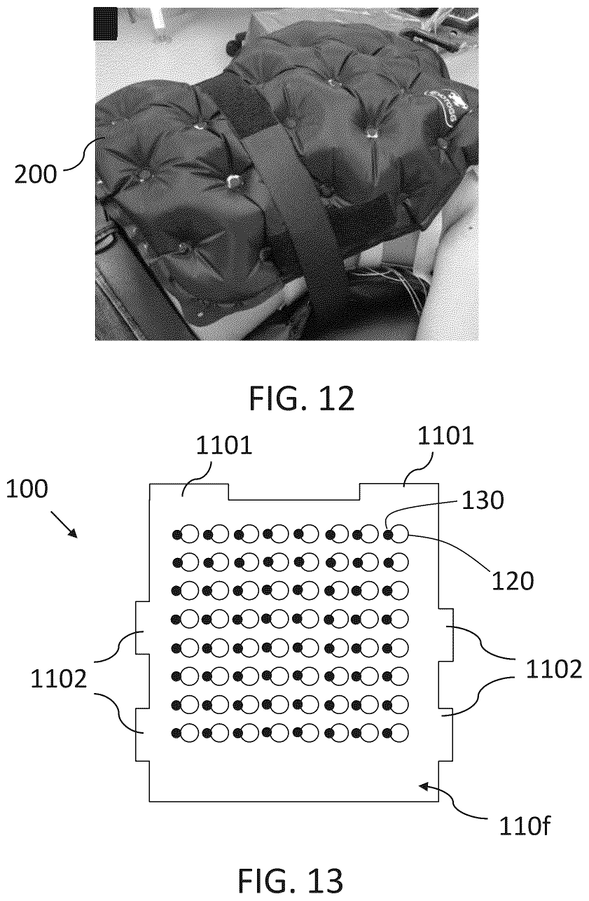

The inflatable gilet 200 comprises an inflatable front panel 201f and an inflatable rear panel 201f that cover the electrodes 120 on the chest and back areas of the vest 100. The front and rear panels 201f, 201r each comprise an interior space 201fi, 201ri that can be inflated via an inflation port or valve 202. The front and rear interior spaces 201fi, 201ri can be fluidly connected, or isolated. In the latter case, each of the panel 201f, 201r includes a separate inflation port or valve 201. The front and rear panels 201f, 201r are further connected by shoulder straps 2011 and side straps 2012, that are preferably adjustable, similar to the electrode vest. The front and rear panels 201f, 201r may define an interior space 201fi, 201ri that, when inflated, have a substantially uniform thickness over the area of the respective panels 201f, 201r. Alternatively, the front and rear panels 201f, 201r may be configured, e.g. with appropriate stitching or welding, to define an interior space 201fi, 201ri with a plurality of fluidly connected inflatable pockets. FIG. 12 shows an image of an example inflatable gilet 200 fitted over the electrode vest 100 shown in FIGS. 9A and 9B.

In various embodiments, the electrode vest 100 comprises fiducial markers 130 substantially co-located with the electrodes 120, as depicted in FIG. 13. The fiducial markers 130 are MR-lucent landmarks that are visible in a CMR image. In this way, the vest 100 to be worn (without the electrical leads 52 connected) during CMR imaging, and the structural data obtained from CMR imaging can then be aligned with the ECG data obtained from the electrode vest 100. The fiducial markers 130 may include a plastic capsule or packet filled with an MR-lucent material (e.g. a fluid). The fiducial markers 130 are preferably designed and configured for optimum patient comfort. The fiducial markers 130 may have a size or width of 1 cm or less. Alternatively, the electrode vest 100 can be removed and replaced by a fiducial marker vest 300 that is a replica of the electrode vest 100 but with fiducial markers 320 in the place of the electrodes 120, as discussed below.

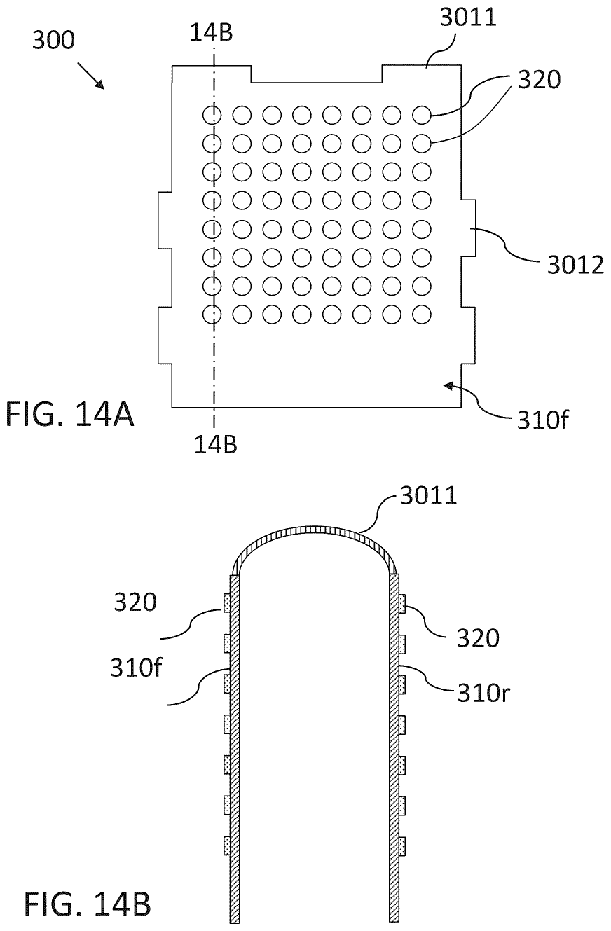

FIG. 14A shows a schematic diagram of a fiducial marker vest 300 according to an embodiment of the present disclosure. FIG. 14B shows a schematic sectional view of the marker vest 300 taken along line 14B-14B. The marker vest 300 comprises flexible front and rear base layers 310f, 310r that are connected via adjustable shoulder straps 3101 and adjustable side straps 1102, as shown. The front base layer 310f covers the patient's chest area, and the rear base layer 310r covers the back area. A first group of the fiducial markers 320 are attached to, and distributed over, the chest area of the front base layer 310f in the same locations as the first group of electrodes 120, and a second group of fiducial markers 320 are attached to, and distributed over, the back area of the rear base layer 310r in the same locations as the second group of electrodes 120. Preferably, the front and rear base layers 310f, 310r are identical in size, shape and material to the front and rear base layers 110f, 110r of the electrode vest 100 to enable accurate alignment of the marker vest 300. For example, the positions of the corners (or other identifiable periphery) of the front and rear base layers 110f, 110r of the electrode vest 100 can be marked on the patient before removal, and the front and rear base layers 310f, 310r of the marker vest 300 can be aligned to those marks to ensure the fiducial markers 320 are in substantially the same locations as the electrodes 120 were.

The fiducial marker vest 300 is CMR compatible and configured for wearing during CMR imaging in the place of the electrode vest 100. The fiducial markers 320 are MR-lucent and visible in a CMR image, allowing structural data obtained from CMR imaging to be aligned with the ECG data obtained from the electrode vest 100. The fiducial markers 320 may include a plastic capsule or packet filled with an MR-lucent material, such as a fluid. The fiducial markers 320 are preferably designed and configured for optimum patient comfort. The fiducial markers 320 may have a size or width of 1 cm or less. The CMR compatible marker vest 300 preferably does not include any metal components. Further, the fiducial marker vest 300 is entirely washable and thereby re-useable. The fiducial marker vest 300 is useful in applications were high throughput and time efficiency is required. Specifically, connecting and disconnecting up to 256 electrical leads 52 can be time consuming. As such, once ECG data is collected, the electrode vest 100 can be quickly removed from the patient (by releasing the straps 1101, 1102) with the electrical leads 52 still attached, and replaced with the marker vest 300 for CMR scanning. This allows the whole process of ECG and CMR data acquisition to be sped up.

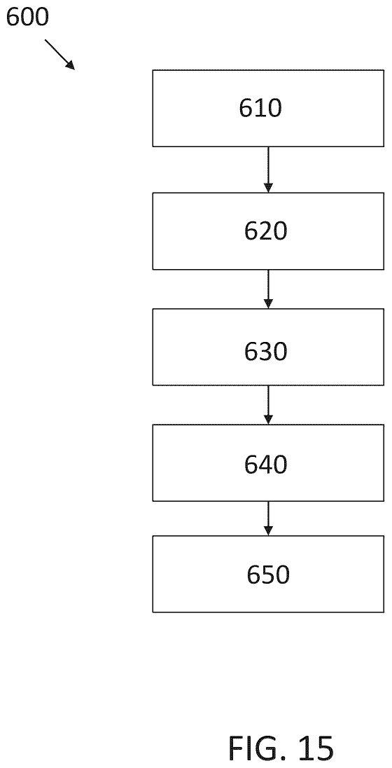

FIG. 15 shows a schematic block diagram of a method 600 of electrocardiographic imaging (ECGi) according to the present disclosure. In step 610, the electrode vest 100 previously described is fitted to the patient. At this stage, the electrical leads 52 may or may not already be connected to the electrodes 120 of the vest 100. If they are not, this step includes connecting or attaching the electrical leads 52 to the electrodes 120 of the vest 100. Preferably, step 610 further comprises fitting the inflatable gilet over the electrode vest 100 and inflating the inflatable gilet 300 to bias the electrodes 120 of the electrode vest 100 towards the patient's skin surface to improve electrode-skin contact. Step 620 comprises acquiring electrophysiological signals from a plurality of locations on the chest skin surface of a patient using the electrode vest 100. This step produces ECG data. This may comprise collecting raw electrical signals from each electrode 120, amplifying the signals, and post-processing. Signal averaging can be performed to improve the quality of the signals from the body surface potentials, such as signal averaging

In step 640, the method further comprises acquiring structural information or data of the patient's heart by performing CMR imaging on the patient. CMR imaging is performed on a magnetic resonance imaging (MRI) system, as is known the art. Examples include 1.5 or 3 Tesla scanner systems with a phased-array chest coil and spine array. The system may be equipped with image reconstruction software. Standardised CMR protocols are known in the art, e.g. as described by C. M. Kramer et al. in “Standardized cardiovascular magnetic resonance imaging (CMR) protocols” 2020 update, Journal of Cardiovascular Magnetic Resonance, (2020), 22(1) 1-8. Acquiring structural information/data for the torso geometry may comprise acquiring HASTE data; a thin contiguously sliced transaxial set of turbo spin echo (TSE) images (e.g. 4 mm thickness with no gaps) across the chest (e.g. approximately 90 slices) using a half-Fourier acquisition single-shot turbo spin echo (HASTE) sequence, as is known in the art. These images localise the fiducial markers and facilitate co-registration during post-processing.

In step 650, one or more maps of electrical activity of the patient's heart are generated based on the electrophysiological signals (ECG data) and the structural information/data, as is known in the art (e.g. see C. Ramanathan et al. Noninvasive electrocardiographic imaging for cardiac electrophysiology and arrythmia. Nat Med. 2004; 10(4):42). The one or more maps may include a bi-atrial map, a biventricular map, and/or a three-dimensional cardiac map. The location of the fiducial markers on the torso corresponding to the electrode positions is combined with the heart-torso geometry obtained from the CMR structural information/data, e.g. the HASTE data, and reconstructed to create epicardial meshes. This can be achieved using commercially available software. Various epicardial electrograms and maps can then be generated. These maps can be used to identify discrete areas of abnormal substrate electrophysiology and correlated with matching substrate abnormalities detected by CMR.

Where the electrode vest 100 comprises a plurality of fiducial markers 320 substantially co-located with the plurality of electrodes 120, step 640 can comprise performing CMR imaging of the patient while wearing the electrode vest 100. In this case, step 640 is preceded by a step 630 of removing the electrical leads 52 from the electrode vest 100 prior to performing CMR imaging. The fiducial markers 320 are captured by the CMR imaging. In this case, the step 650 of generating the one or more maps of electrical activity of the patient's heart based on the electrophysiological signals and the structural information comprises aligning the electrophysiological signals (or ECG data) obtained from the electrodes 120 of the electrode vest 100 to the structural information using the captured fiducial markers in the CMR imaging.

Optionally, the step 640 of acquiring structural information can comprise performing CMR imaging of the patient while wearing the fiducial marker vest 300 in the place of the electrode vest 100. In this case, step 630 comprises removing the electrode vest 100 and fitting the fiducial marker vest 300 to the patient, such that the fiducial markers 320 are in the same prior locations as the electrodes 120. This may include a step of marking the position of the corners (or other identifiable peripheral feature) of the front and rear base layers 110f, 110r of the electrode vest 100 on the patient's skin prior to its removal, and aligning the front and rear base layers 310f, 310r of the marker vest 300 to the positions marked on the patient's skin. The fiducial markers 320 are captured by the CMR imaging performed in step 640. The step 650 then comprises aligning the electrophysiological signals (ECG data) obtained from the electrodes 120 of the electrode vest 100 to the structural information using the captured fiducial markers 320 in the CMR imaging.

From reading the present disclosure, other variations and modifications will be apparent to the skilled person. Such variations and modifications may involve equivalent and other features which are already known in the art, and which may be used instead of, or in addition to, features already described herein.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub-combination.

For the sake of completeness it is also stated that the term “comprising” does not exclude other elements or steps, the term “a” or “an” does not exclude a plurality, and any reference signs in the claims shall not be construed as limiting the scope of the claims.

1. An electrocardiographic imaging (ECGi) electrode vest comprising:
a flexible base layer configured to cover at least a patient's chest area; and
a plurality of dry contact electrodes attached to the flexible base layer and distributed over at least a chest area of the flexible base layer to acquire electrophysiological signals from the patient's skin surface;
wherein each electrode comprises a sensing portion having an exposed sensing surface on an interior skin facing side of the electrode to contact the user's skin surface when the vest is worn, and an exposed connector portion on an exterior side of the electrode for removably connecting with an electrical lead of an ECGi measurement apparatus.

2. The electrode vest of claim 1, wherein the electrode vest does not include any electrical leads or traces for conducting the electrophysiological signals from the electrodes.

3. The electrode vest of claim 1, wherein at least part of the electrodes are formed of or comprise a conductive polymer material.

4. The electrode vest of claim 1, wherein the connector portion comprises a protrusion for removably connecting with an electrical lead of an ECGi measurement apparatus.

5. The electrode vest of claim 1, wherein the base layer comprises a plurality of openings, and wherein each electrode extends through a respective one of the plurality of openings and comprises first and second flanges that extend over opposite sides of the base layer to grip the base layer therebetween.

6. The electrode vest of claim 5, wherein each electrode comprises an electrode body with a first part and a second part, the first part including the first flange and the second part including the second flange, the first and second parts being connectable to each other through a respective opening in the base layer to attach the electrode body to the base layer, optionally or preferably, wherein the first and second parts are connectable in a snap-fit manner.

7. The electrode vest of claim 6, wherein each electrode comprises a third part disposed on or embedded in a skin facing side of the base layer adjacent the respective opening and having an exposed sensing surface to contact the user's skin surface when the vest is worn, the third part electrically contacting the electrode body when the first and second parts are connected through the respective opening to extend the sensing surface of the electrode.

8. The electrode vest of claim 1, wherein the electrodes are uniformly distributed in an array over at least the chest area of the base layer.

9. The electrode vest of claim 1, further comprising a plurality of fiducial markers substantially co-located with the plurality of electrodes.

10. An electrocardiographic imaging (ECGi) kit, comprising:
the ECGi electrode vest of claim 1; and
an inflatable gilet configured to be worn over the electrode vest during electrophysiological signal acquisition to improve the skin-electrode contact.

11. The kit of claim 10, wherein the inflatable gilet is configured, when worn over the electrode vest and inflated, to exert a compressive force on the electrode vest to bias the electrodes towards a patient's skin surface.

12. The kit of claim 10, further comprising a CMR compatible fiducial marker vest for wearing during CMR imaging, the fiducial marker vest comprising a flexible base layer configured to cover at least a patient's chest area, and a plurality of fiducial markers distributed over a chest area of the base layer in the same locations as the electrodes of the electrode vest.

13. The kit of claim 10, further comprising a plurality of electrical leads, each electrical lead comprising an electrode connector head at one end of thereof configured to removably connect with a respective connector portion of a respective electrode of the electrode vest for transmitting electrophysiological signals to an ECGi measurement apparatus.

14. An electrocardiographic imaging (ECGi) system, comprising:
the ECGi electrode vest of claim 1; and
an ECGi measurement apparatus comprising: a data acquisition system with a plurality of measurement channels; and a plurality of electrical leads, each electrical lead comprising an electrode connector head at one end thereof configured to removably connect to a respective connector portion of a respective electrode of the electrode vest for transmitting electrophysiological signals to a respective measurement channel of the data acquisition system.

15. The system of claim 14, wherein each connector portion of each electrode comprises a protrusion, and wherein each electrode connector head comprises one of: a mechanical clip configured to releasably engage the protrusion, and a female connector configured to receive and frictionally and/or mechanically engage the protrusion.

16. The system of claim 14, wherein each electrode connector head comprises a signal pre-amplifier.

17. The system of claim 14, further comprising an inflatable gilet configured to be worn over the electrode vest during electrophysiological signal acquisition to improve the skin-electrode contact.

18. A method of electrocardiographic imaging (ECGi), comprising:
acquiring electrophysiological signals from a plurality of locations on the chest skin surface of a patient using the ECGi electrode vest of claim 1;
acquiring structural information of the patient's heart by performing cardiovascular magnetic resonance (CMR) imaging on the patient;
generating one or more maps of electrical activity of the patient's heart based on the electrophysiological signals and the structural information.

19. The method of claim 18, wherein the step of acquiring electrophysiological signals comprises:
fitting the electrode vest to the patient;
fitting an inflatable gilet over the electrode vest; and
inflating the inflatable gilet to bias the electrodes of the electrode vest towards the patient's skin surface.

20. The method of claim 19, wherein the step of acquiring electrophysiological signals further comprises attaching a plurality of electrical leads to the electrodes.

21. The method of claim 20, wherein the electrode vest comprises a plurality of fiducial markers substantially co-located with the plurality of electrodes, and the step of acquiring structural information comprises:
removing the electrical leads from the electrode vest; and
performing CMR imaging on the patient, wherein the fiducial markers are captured by the CMR imaging.

22. The method of claim 21, wherein the step of generating the one or more maps of electrical activity of the patient's heart based on the electrophysiological signals and the structural information comprises:
aligning the electrophysiological signals obtained from the electrodes of the electrode vest to the structural information using the captured fiducial markers in the CMR imaging.

23. The method of claim 18, wherein the step of acquiring structural information comprises:
removing the electrode vest and fitting a fiducial marker vest to the patient, the fiducial marker vest comprising a flexible base layer configured to cover at least the patient's chest area, and a plurality of fiducial markers distributed over the chest area of the base layer in the same locations as the electrodes of the electrode vest; and
performing CMR imaging on the patient, wherein the fiducial markers are captured by the CMR imaging.

24. The method of claim 23, wherein the step of generating the one or more maps of electrical activity of the patient's heart based on the electrophysiological signals and the structural information comprises:
aligning the electrophysiological signals obtained from the electrodes of the electrode vest to the structural information using the captured fiducial markers in the CMR imaging.
